SEQUENCE LISTING (6 sequences)

SEQ ID NO: 1 - DNA, 1546 bp, Arabidopsis thaliana
atccttcaccaatcaaaaccttctcatgtcttcttctctcctcgacctttgaggtggaaaattaaatatattcccttagctttttttctcctttagttttcttcttcttcttgagttttttttcttttgatcctctctaatttccttgttgattcatcgactagatctaattcttctcacaaaagactgagtgtgttctttctttcaaatctttcaaaaactagggtttttactgtcttgaaatcatatttattcttctaaatttagcaaaaagaacacgatttactttccatttcagtcgtcttgtcactctctctctcttctttaaagtctccctttttagcaaaaattctctctctcacaaaatttatttcctctggcttcttcttcctcctcctccatctcttctctttactctctctttaatcatctctcattcttgaatcttgatccatcaaaatcaatcccgttctcgaaagatccattaaaatcaaaacctaagctctctctcttgcttctagggtttttttgttcgttgtgatggcgagagaaaagattcagatcaggaagatcgacaacgcaacggcgagacaagtgacgttttcgaaacgaagaagagggcttttcaagaaagctgaagaactctccgttctctgcgacgccgatgtcgctctcatcatcttctcttccaccggaaaactgttcgagttctgtagctccagcatgaaggaagtcctagagaggcataacttgcagtcaaagaacttggagaagcttgatcagccatctcttgagttacagctggttgagaacagtgatcacgcccgaatgagtaaagaaattgcggacaagagccaccgactaaggcaaatgagaggagaggaacttcaaggacttgacattgaagagcttcagcagctagagaaggcccttgaaactggtttgacgcgtgtgattgaaacaaagagtgacaagattatgagtgagatcagcgaacttcagaaaaagggaatgcaattgatggatgagaacaagcggttgaggcagcaaggaacgcaactaacggaagagaacgagcgacttggcatgcaaatatgtaacaatgtgcatgcacacggtggtgctgaatcggagaacgctgctgtgtacgaggaaggacagtcgtcggagtctattactaacgccggaaactctaccggagcgcctgttgactccgagagctccgacacttcccttaggctcggcttaccgtatggtggttagagatggaacaattcaaagaagttgatggagtgaggagagtaatgtaaatctttttaactcggtagtaacaagagacaatgtctaagtagtgaattctcaaatgtttgtgtaagtttctgcctatggaagaggctttcatttttatgattttcactatgtatgatctctcttcactgcatttctggttagtaacgcttgtcaccgataaactttctcgttatggaaagttagaatataaaatattgtagaattttgattatcgatatactcttggttagggatc

SEQ ID NO: 2 - protein, 240 aa, Arabidopsis thaliana
MAREKIQIRKIDNATARQVTFSKRRRGLFKKAEELSVLCDADVALIIFSSTGKLFEFCSSSMKEVLERHNLQSKNLEKLDQPSLELQLVENSDHARMSKEIADKSHRLRQMRGEELQGLDIEELQQLEKALETGLTRVIETKSDKIMSEISELQKKGMQLMDENKRLRQQGTQLTEENERLGMQICNNVHAHGGAESENAAVYEEGQSSESITNAGNSTGAPVDSESSDTSLRLGLPYGG

SEQ ID NO: 3 - DNA, 28 bp, Artificial Sequence
  primer
gtggtcgacactttttattttactctgg

SEQ ID NO: 4 - DNA, 28 bp, Artificial Sequence
  primer
ggatccgcaccaccatacggtaagctgc

SEQ ID NO: 5 - DNA, 25 bp, Artificial Sequence
  primer
tgaacactaacatgattgaatgaca

SEQ ID NO: 6 - DNA, 21 bp, Artificial Sequence
  primer
gatcttgaacaaacaggtggt

(19) United States
(12) Patent Application Publication
(10) Pub. No.: US 2008/0263726 A1
(43) Pub. Date: Oct. 23, 2008

(54) SVP gene controlling flowering time of plants

(75) Inventors: Jong Seob Lee, Seoul (KR); Jeong Hwan Lee, Seoul (KR); Seong Jeon Yoo, Seoul (KR); Soo Hyun Park, Goyang-si (KR); Il doo Hwang, Pohang-si (KR); Ji Hoon Ahn, Seoul (KR); Yang Do Choi, Seoul (KR)

Correspondence Address: GREER, BURNS & CRAIN, 300 S WACKER DR, 25TH FLOOR, CHICAGO, IL, 60606, US

(73) Assignee: Seoul National University Industry Foundation, Seoul (KR)

(21) Appl. No.: 12/012,547

(22) Filed: Feb. 4, 2008

(30) Foreign Application Priority Data
Feb. 6, 2007 (KR) 10-2007-0012375

(51) Int. Cl. A01H 1/00; C07K 14/415; C07H 21/00; C12Q 1/68; C12N 15/63; A01H 5/00

(52) U.S. Cl. 800/278; 530/379; 536/23.6; 435/320.1; 800/298; 435/6

(57) ABSTRACT

The present invention relates to SVP protein which controls the flowering time of plants originating from Arabidopsis, a gene encoding SVP protein, a recombinant vector comprising said gene, a plant transformed with said recombinant vector, a method of controlling flowering time of plants by using said gene, and a method of searching a protein or a gene which controls the flowering time of plants by using said SVP protein or said gene encoding the same.

### BRIEF DESCRIPTION OF THE DRAWINGS

FIG. 1. Role of SVP in the temperature-dependent control of flowering in Arabidopsis. (A) Flowering time of a group of flowering time mutants at 23° C. and 16° C. under long-day conditions. The numbers listed above the genotypes denote the ratios of flowering time at 16° C. and 23° C. (16° C./23° C.). Error bars indicate the standard deviation. The inset shows wild-type Columbia (Col) plants and svp-32 plants grown at 23° C. and 16° C. (B) Effects of low temperature on SVP expression in wild-type plants (Col). SVP, FLC, and FT expression levels were measured by real-time PCR in the leaf of 10-d-old seedlings grown at the indicated temperatures. Tubulin was employed as an internal control. (C) Histochemical analysis of 10-d-old seedlings of SVP::GUS and FT::GUS plants grown at 23° C. and 16° C. Scale bars, 500 μm. (D) Nuclear localization of SVP-GFP fusion protein in onion epidermal cells incubated at 16° C. and 23° C. The nucleus is indicated by an arrow. 4′-6-Diamidino-2-phenylindole (DAPI) was used for nuclear staining. Scale bars, 10 μm.

FIG. 2. Genetic interaction of SVP with FCA, FVE, and FLC. (A) Flowering time of the svp-32 fca-9, and svp-32 fve-3 double mutants at 23° C. and 16° C. under long-day conditions. The numbers listed below denote the ratios of flowering time (16° C./23° C.). (B) Effects of fca and fve mutations on SVP expression in 10-d-old seedlings. (C) SVP expression in 10-d-old seedlings of loss- and gain-of-function alleles of FLC. (D) FLC expression in 10-d-old seedlings of the loss- and gain-of-function mutants of SVP. (E) Flowering time of mutants harboring various combinations of svp, fri, and flc mutations at 23° C. and 16° C. SVP fri FLC and svp fri FLC indicate wild-type Columbia plants and svp-32 plants, respectively. The numbers listed below denote the ratios of flowering time (16° C./23° C.).

FIG. 3. Role of SVP as an FT repressor. (A) Time-course expression of FT in wild-type (Col) and svp-32 plants at 23° C. and 16° C. FT expression level was monitored in 6-, 8-, 10-, 12-, and 14-d-old seedlings. (B) pFT::GUS expression patterns in 10-d-old seedlings of wild-type (Col) and svp-32 plants at 23° C. Scale bars, 500 μm. (C) Flowering time of svp-32 ft-10, svp-32 35S::FT, and ft-10 soc1-2 double mutants at 23° C. and 16° C. The numbers listed below denote the flowering time ratios (16° C./23° C.).

FIG. 4. Binding of SVP protein to the vCArG III in the FT promoter. (A) A chromatin immunoprecipitation assay using protoplasts transfected with SVP-HA and FLC-HA constructs. The location of six variants of CArG motifs (vCArG I to VI) identified in a 1.8-kb FT promoter and the different fragments analyzed by PCR are represented. A four-fold dilution series of the input DNA was used as a semi-quantitative standard. Input, total input chromatin DNA; HA, DNA selected using HA antibodies; Myc, DNA selected using Myc antibodies. (B) The effects of SVP-HA protein on the FT promoter activities. A schematic representation of the reporters and the effectors used in this assay is shown. Variants of CArG motifs are shaded in grey and mutations introduced in vCArG motifs are indicated in lowercase. m3FT::LUC indicates the FT::LUC construct harboring a mutated vCArG III.

### DETAILED DESCRIPTION OF THE INVENTION

### Background Art

The present invention relates to a gene which controls flowering time of plants and a method of controlling flowering time of plants using the same. More specifically, the present invention relates to SVP protein which controls the flowering time of plants originating from Arabidopsis, a gene encoding SVP protein, a recombinant vector comprising said gene, a plant transformed with said recombinant vector, a method of controlling flowering time of plants by using said gene, and a method of searching a protein or a gene which controls the flowering time of plants by using said SVP protein or said gene encoding the same.

In general, flowering time of plants are affected by environmental conditions or has been decided genetically. Factors that can affect flowering time of plants include an external environmental condition such as light and temperature and an internal condition such as a signal for development, etc. For Arabidopsis, it has been long recognized that its flowering time is changed by photoperiod; i.e., the flowering time is accelerated under long-day condition while it is delayed under short-day condition. For example, it has been known that flowering time of plants is significantly delayed when plants are grown at low temperature. It has been believed that such influence by temperature is due to a slow-down of overall metabolism rate.

Plants are sessile organisms and are, consequently, exposed to a wide variety of environmental stresses, both abiotic and biotic, exerted by their surroundings. The most common of these is temperature. Within the range of temperatures tolerable to plants, the response to low temperature, particularly near-freezing temperature, is well understood. Plants have evolved a number of adaptive mechanisms to meet the challenge of low temperature. In Arabidopsis, flowering is accelerated by prolonged exposure to cold, a process called vernalization. The epigenetic silencing of the FLOWERING LOCUS C (FLC) is central to the vernalization process, and this silencing has been attributed to the activities of the VERNALIZATION1 (VRN1), VERNALIZATION2 (VRN2), and VERNALIZATION INSENSITIVE3 (VIN3) genes.

Analyses of mutant plants have identified C-Repeat binding factor (CBF)-dependent and -independent signaling pathways in cold acclimation, suggesting that plants employ distinct mechanisms to respond to low temperature (Sharma, P. et al., 2005 Bioessays 27: 1048-1059). In case of Arabidopsis, expression of many genes is induced during an adaptation period to low temperature. Specifically, there are expressions of genes including RD29A (also known as COR78 or LT178), KIN1, KIN2, COR15A and COR47 (or RD17), etc.

There is increasing concern about the potential impact of global temperature changes, which significantly affect ambient temperature, on plant development. Several lines of evidence suggest that the recently observed alterations in the flowering times of many plant species and the increase in plant respiration rates are closely associated with these changes in ambient temperature (Fitter, A. H. and Fitter, R. S. 2002. Science 296: 1689-1691; Atkin, O.K. and Tjoelker, M. G. 2003. Trends Plant Sci. 8: 343-351).

According to U.S. Pat. No. 6,225,530, FT (flowering locus T) gene which is isolated from Arabidopsis and controls flowering time of plants, a polypeptide encoded by FT and a method of modulating flowering time in plants using FT gene are disclosed.

Although a great deal of progress has been made in our understanding of the regulation of plant development by low temperature, less is currently known about the molecular mechanisms underlying the responses of plants to changes in ambient temperature.

Under the circumstances, while studying the genes that are related to a new mechanism for controlling the flowering time in Arabidopsis, inventors of the present invention found that SHORT SHORT VEGETATIVE PHASE (SVP) gene mediates ambient temperature signaling process in Arabidopsis and the SVP-mediated control of FLOWERING LOCUS T (FT) gene expression can modulate the timing of the developmental transition to flowering phase in response to changes in the ambient temperature, and therefore completed the present invention.

### Technical Subject to be Achieved by the Invention

Therefore, one object of the present invention is to provide SVP protein, which controls flowering time of plants.

Another object of the present invention is to provide a gene, which encodes said SVP protein for controlling flowering time of plants.

Another object of the present invention is to provide a recombinant vector comprising said gene for controlling flowering time of plants.

Another object of the present invention is to provide a plant transformed with said vector.

Another object of the present invention is to provide a method for controlling flowering time of plants by using said gene for controlling flowering time of plants.

Still another object of the present invention is to provide a method for searching a protein or a gene which can control flowering time of plants by using said gene and protein for controlling flowering time of plants.

### Constitution of the Invention

In order to achieve the purposes described above, the present invention provides a protein named SHORT VEGETATIVE PHASE (SVP), which consists of amino acid sequence as described in SEQ ID NO: 2 and controls flowering time of plants originating from Arabidopsis.

The scope of the protein which controls flowering time of plants according to the present invention includes a protein having an amino acid sequence described in SEQ ID NO: 2 that is isolated from Arabidopsis and functional equivalents of said protein. The term “functional equivalent” means that, as a result of addition, substitution or deletion of amino acid residues, it has a amino acid sequence with at least 70%, preferably at least 80%, more preferably at least 90%, still more preferably at least 95% homology with the amino acid sequence of SEQ ID NO: 2, thus indicating a protein which has substantially the same physiological activity as the protein represented by SEQ ID NO: 2.

The term “substantially the same physiological activity” means that, when overexpressed in plants, it can delay the flowering in plants. More preferably, it can be an activity by which flowering time of plants is controlled in response to a change in ambient temperature. Preferably, SVP protein for controlling flowering time of plants according to the present invention has a amino acid sequence as described in SEQ ID NO: 2. SVP protein according to the present invention can be extracted from natural source (e.g., plant cell) or obtained either by an expression of recombinant nucleotides which encodes SVP protein or by a chemical synthetic method.

The present invention further provides a gene which encodes said SVP protein for controlling flowering time of plants. The gene for controlling flowering time of plants according to the present invention includes both of genomic DNA and cDNA which encodes SVP protein. Preferably, said gene of the present invention may comprise a nucleotide sequence represented by SEQ ID NO: 1. Moreover, said gene of the present invention can be directly connected to CArG motif of FT (flowering locus T) gene, which is a gene for promoting flowering in plants, to inhibit the expression of FT gene, resulting in the flowering time control in plants.

Variants of said nucleotide sequence are also within the scope of the present invention. Specifically, said gene may comprise a nucleotide sequence with at least 70%, preferably at least 80%, more preferably at least 90%, still more preferably at least 95% homology with the nucleotide sequence of SEQ ID NO: 1. Said “sequence homology %” for a certain polynucleotide is determined by comparing two nucleotide sequences that are optimally arranged with a region to be compared. In this regard, a part of the polynucleotide sequence in a region to be compared may comprise an addition or a deletion (i.e., a gap) compared to a reference sequence (without any addition or deletion) relative to the optimized arrangement of the two sequences.

The present invention further provides a recombinant vector comprising a gene for controlling flowering time of plants according to the present invention. Said recombinant vector is preferably a recombinant plant expression vector.

The term “recombinant” indicates a cell which replicates a heterogeneous nucleotide or expresses said nucleotide, a peptide, a heterogeneous peptide, or a protein encoded by a heterogeneous nucleotide. Recombinant cell can express a gene or a gene fragment in a form of a sense or antisense, that are not found in natural state of cell. In addition, a recombinant cell can express a gene that is found in natural state, provided that said gene is modified and re-introduced into the cell by an artificial means.

The term “vector” is used herein to refer DNA fragment (s) and nucleotide molecules that are delivered to a cell. Vector can replicate DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operatively-linked coding sequence in a specific host organism. A promoter, an enhancer, a termination signal and a polyadenylation signal that can be used for an eukaryotic cell are all publicly well known.

A preferred example of plant expression vector is Ti-plasmid vector which can transfer a part of itself, i.e., so-called T-region, to a plant cell when the vector is present in an appropriate host such as Agrobacterium tumefaciens. Other types of Ti-plasmid vector (see, EP 0 116 718 B1) are currently used for transferring a hybrid gene to protoplasts that can produce a new plant by appropriately inserting a plant cell or hybrid DNA to a plant genome. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded plant virus, and a viral vector which can be originated from Gemini virus, etc., for example a non-complete plant viral vector. Use of said vector can be advantageous especially when a plant host cannot be appropriately transformed.

Expression vector would comprise at least one selective marker. Said selective marker is a nucleotide sequence having a property which allows a selection based on a common chemical method. Any kind of gene that can be used for the differentiation of transformed cells from non-transformed cell can be a selective marker. Example includes, a gene resistant to herbicide such as glyphosate and phosphintricin, and a gene resistant to antibiotics such as kanamycin, G418, bleomycin, hygromycin, and chloramphenicol, but not limited thereto.

For the plant expression vector according to one embodiment of the present invention, a promoter can be any of CaMV 35S, actin, ubiquitin, pEMU, MAS or histone promoter, but not limited thereto. The term “promoter” means a DNA molecule to which RNA polymerase binds in order to initiate its transcription and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states. Since a transformant can be selected with various mechanisms at various stages, a constitutive promoter can be preferable for the present invention. Therefore, a possibility for choosing a constitutive promoter is not limited in the present invention.

For the above-described terminator, any conventional terminator can be used for the present invention. Example includes, nopaline synthase (NOS), rice α-amylase RAmyl A terminator, phaseoline terminator, and a terminator for optopine gene of Agrobacterium tumefaciens, etc., but are not limited thereto. Regarding the necessity of terminator, it is generally known that such region can increase a reliability and an efficiency of transcription in plant cells. Therefore, the use of terminator is highly preferable in view of the contexts of the present invention.

The present invention furthermore provides a plant that is transformed with the recombinant vector according to the present invention. Plant transformation means any method by which DNA is delivered to a plant. Such transformation method does not necessarily have a period for regeneration and/or tissue culture. Transformation of plant species is now quite general not only for dicot plants but also for monocot plants. In principle, any transformation method can be used for introducing a hybrid DNA of the present invention to an appropriate progenitor cells. It can be appropriately selected from a calcium/polyethylene glycol method for protoplasts (Krens, F. A. et al., 1982, Nature 296, 72-74; Negrutiu I. et al., June 1987, Plant Mol. Biol. 8, 363-373), an electroporation method for protoplasts (Shillito R. D. et al., 1985 Bio/Technol. 3, 1099-1102), a microscopic injection method for plant components (Crossway A. et al., 1986, Mol. Gen. Genet. 202, 179-185), a particle bombardment method for various plants components (DNA or RNA-coated) (Klein T. M. et al., 1987, Nature 327, 70), or a (non-complete) viral infection method in Agrobacterium tumefaciens mediated gene transfer by plant invasion or transformation of fully ripened pollen or microspore (EP 0 301 316), etc. A method preferred in the present invention includes Agrobacterium mediated DNA transfer. In particular, so-called binary vector technique as disclosed in EP A 120 516 and U.S. Pat. No. 4,940,838 can be preferably adopted for the present invention.

The term “plant cell” that is used for the plant transformation according to the present invention can be any plant cell. The plant cell can be a cultured cell, a cultured tissue, a cultured organ, or a whole plant, preferably a cultured cell, a cultured tissue or a cultured organ, and more preferably any form of a cultured cell.

The term “plant tissue” includes either differentiated or undifferentiated plant tissue, including root, stem, leaf, pollen, seed, cancerous tissue and cells having various shape that are used for culture, i.e., single cell, protoplast, bud and callus tissue, but not limited thereto. Plant tissue can be in planta or in a state of organ culture, tissue culture or cell culture.

The present invention furthermore provides a method for controlling flowering time of plants by using a gene which controls flowering time of plants according to the present invention. More specifically, the present invention provides a method for controlling flowering time of plants, characterized in that flowering time of plants is delayed by overexpressing SVP gene in plants or it is accelerated by inhibiting the expression of SVP gene in plants.

For carrying out a process for overexpressing SVP gene in plants, SVP gene can be introduced to a plant with SVP gene or a plant without SVP gene. In this connection, the term “gene overexpression” means that SVP gene is expressed above the level that is normally found in a wild type plant. For introducing SVP gene into a plant, an expression vector comprising SVP gene that is under regulation of a promoter can be used to transform the plant. In this regard, the promoter is not specifically limited as long as it can allow the overexpression of an inserted gene in plants. Non-limiting example of such promoter includes, 35S RNA and 19S RNA promoter of CaMV; whole-length transcription promoter originating from figwort mosaic virus (FMV) and coat protein promoter of TMV. In addition, in order to achieve SVP gene overexpression in monocot plants or wood plants, ubiquitin promoter can be used.

For a method of inhibiting the expression of SVP gene in plants, various methods known in the pertinent art can be used. The term “inhibition of gene expression” includes the inhibition of gene transcription as well as the inhibition of its translation into a protein. Furthermore, the inhibition includes not only the complete termination of gene expression but also the reduction in the expression.

For inhibiting an expression of a certain endogenous gene in plants, the use of an antisense molecule is the most typical. As a mechanism for an antisense molecule to inhibit an expression of target gene, there are several ways as follows; an inhibition of transcription initiation by forming a triple strand, an inhibition by forming a hybrid at a site wherein a local open loop is formed by RNA polymerase, an inhibition by forming a hybrid with RNA that is responsible for carrying out translation, an inhibition of splicing by forming a hybrid at joint position between intron and exon, an inhibition of splicing by forming a hybrid at a site wherein a splisome is formed, an inhibition of transport from a nucleus to cytoplasm by forming a hybrid with mRNA, and an inhibition of translation initiation by forming a hybrid at a site to which initiators for translation bind, etc. Said methods prohibit a transcription, a splicing or a translation process, eventually inhibiting the expression of a target gene.

An antisense molecule used in the present invention can inhibit the expression of a target gene based on any kind of mechanisms. Exemplary antisense molecules include triple-strand forming oligonucleotide, ribozyme, RNAi, and an antisense nucleotide, etc. The triple-strand forming oligonucleotide wraps around DNA to form a triple-strand, thus resulting an inhibition of transcription initiation (Maher et al., Antisense Res. and Dev., 1(3):227, 1991; Helene, C., Anticancer Drug Design, 6(6):569, 1991). Ribozyme is a RNA molecule which can specifically digest a single-stranded RNA. Ribozyme can be artificially engineered so that it can recognize a specific nucleotide sequence included in RNA molecule and carry out a site-specific digestion (Cech, J. Amer. Med. Assn., 260:3030, 1998). Therefore, main advantage of this method is that, being specific to certain nucleotide sequence, it can specifically inactivate mRNA molecules comprising such certain sequences. RNAi method is based on an inhibition at transcription level or post-transcription level by using a small RNA molecule having hairpin shape, of which action is specific to the nucleotide sequence (Mette et al., EMBO J., 19: 5194-5201, 2000). An antisense nucleotide is a DNA or RNA molecule characterized in that at least a part of its sequence is complementary to a specific mRNA molecule (Weintraub, Scientific American, 262:40, 1990). An antisense nucleotide hybridizes to a corresponding mRNA comprised in a cell to form a double-stranded molecule. As a result, translation of the mRNA becomes inhibited (Marcus-Sakura, Anal. Biochem., 172:289, 1988).

The method for controlling flowering time of plants according to the present invention can be applied for producing flowers and seeds in short period of time by inhibiting the expression of SVP gene and thus accelerating flowering time of horticultural plants, or for increasing a productivity of useful plant parts that can be obtained from agricultural crops by overexpressing SVP gene and delaying flowering time of the crops so as to achieve a continuous induction of vegetative growth.

Said plant can be food crops including rice, wheat, barley, corn, soybean, potato, red bean, oat and millet; vegetable crops including Arabidopsis thaliana, Chinese cabbage, radish, hot pepper, strawberry, tomato, watermelon, cucumber, cabbage, melon, pumpkin, scallion, onion and carrot; special crops including ginseng, tobacco, cotton, sesame, sugar cane, sugar beet, wild sesame, peanut and rapseed; fruits including apple, pear, date, peach, kiwi, grape, orange, persimmon, plum, apricot and banana; flowers including rose, gladiolus, gerbera, carnation, chrysanthemum, lily, and tulip; and feed crops including rye grass, red clover, orchard grass, alfalfa, tall fescue, and perennial rye grass.

The present invention still furthermore provides a transformed plant having controlled flowering time, which is produced by the above-described method of the present invention.

The plant having controlled flowering time can be obtained by a method publicly known in the pertinent art, e.g., sexual propagation method or asexual propagation method. More specifically, the plant of the present invention can be obtained by a sexual propagation method by which seeds are first produced by flower pollination and then propagation is carried out by using them. Moreover, it can be obtained by an asexual propagation method by which a plant is first transformed with a recombinant vector comprising SVP gene of the present invention and then callus is induced, roots are formed and adapted to soils according to a conventional method. In other words, a fragment of the plant which has been transformed with the recombinant vector comprising SVP gene is placed in an appropriate culture medium that is known in the pertinent art and cultured under an appropriate condition to induce formation of callus, and then it is transferred to a hormone-free medium for culture right after the plant shoots are formed. Approximately two weeks later, the shoots are transferred to a new medium for growing roots in order to induce them. Once the roots are induced, they are transplanted and adapted to soils to obtain the plants having controlled flowering time. According to the present invention, the transformed plant includes not only a whole plant but also a tissue, a cell and a seed that can be obtained therefrom.

Furthermore, the present invention provides a method of searching a protein or a gene of which flowering time is controlled, by carrying out an analysis using SVP protein or the gene encoding the same, wherein said analysis is selected from a group consisting of DNA chip method, protein chip method, polymerase chain reaction (PCR), Northern blot analysis, Southern blot analysis, enzyme-linked immunosorbent assay (ELISA) and 2D gel analysis. In addition, by determining a substance which binds to the gene of the present invention or a substance which can inhibit or activate the expression of SVP gene, said method of the present invention can be used as a tool for studying the flowering in plants of interest. More specifically, the analysis can be carried out by using various methods including DNA chip method, protein chip method, polymerase chain reaction (PCR), Northern blot analysis, Southern blot analysis, enzyme-linked immunosorbent assay (ELISA) and 2D gel analysis, etc.

The present invention will now be described in greater detail with reference to the following examples. However, it is only to specifically exemplify the present invention and in no case the scope of the present invention is limited by these examples.

### EXAMPLE

### Materials and Methods

### Plant Materials, Growth Conditions, and Measurement of Flowering Time

All mutations used in this study were in the Columbia (Col) background, unless otherwise noted. svp-31 (SALK_{—}026551) and svp-32 (SALK_{—}072930), both T-DNA insertion lines of SVP, were obtained from the Arabidopsis Biological Resource Center (ABRC) (Alonso, J. M. et al., 2003. Science 301: 653-657). To confirm the T-DNA insertion sites of these alleles, we sequenced the PCR products amplified using left border primers and gene-specific primers. SVP overexpressor plants obtained from H. Sommer have been described previously (Masiero, S. et al., 2004. Development 131: 5981-5990). The plants were grown in soil or MS medium at 23° C. or 16° C. under long-day (LD) conditions [16/8 h (light/dark)] with light provided at an intensity of 120 μmol m⁻²s⁻¹. The homozygosity of the double mutants was verified via PCR genotyping. The flowering times of the plants are expressed as the total number of primary leaves of at least 12 plants.

### Expression Analysis

Expression levels of the flowering time genes were determined via semi-quantitative reverse transcriptase-meditated PCR or real-time PCR. Total RNA was extracted using Trizol reagent (Invitrogen, Carlsbad, Calif.), and 1 μg of total RNA was used to synthesize the complementary DNA. The primer sequences and amplification conditions are available on request. The real-time PCR analysis was performed using an ABI PRISM 7900HT sequence detection system (Applied Biosystems, Foster City, Calif.), and expression levels were normalized against that of tubulin. For the histochemical GUS analysis, we generated a SVP::GUS translational fusion construct. The 4.9-kb SVP genomic region was amplified using JH2929 (5′-GTGGTCGACACTTTTTATTTTACTCTGG-3′) (SEQ ID NO: 3) and JH2985 (5′-GGATCCGCACCACCATACGGTAAGCTGC-3′) (SEQ ID NO: 4) and then fused with the GUS reporter gene. FT::GUS plants were obtained from K. Goto. SVP cDNA-GFP chimeric constructs were used as a reporter to examine the localization pattern of SVP. To generate the 35S promoter-driven SVP cDNA-GFP construct, the GFP sequence was in-frame fused to the C-terminal region of a 35S::SVP chimeric plasmid. A particle bombardment system (PDS-1000/He; Bio-Rad, Hercules, Calif.) was utilized for the delivery of DNA-coated tungsten particles into onion epidermal cells. After 24 hours of incubation at 23° C. or 16° C., the subcellular localization pattern was observed under a fluorescence microscope (Carl Zeiss).

### Chromatin Immunoprecipitation (ChIP) Assay

ChIP assays were conducted as described (Tang, W. and Perry, S. E. 2003. J. Biol. Chem. 278: 28154-28159) with minor modifications. The Arabidopsis protoplasts were transfected with either SVP cDNA fused to HA tags or FLC cDNA fused to HA tags and then incubated for 24 hours at room temperature. The expressions of the SVP-HA and FLC-HA proteins were determined by protein blots using extracts from the protoplasts. After formaldehyde fixation, the chromatin of the protoplasts was isolated and sheared via sonication. Mouse anti-HA antibodies (Santa Cruz Biotech, Santa Cruz, Calif.) or anti-Myc 9B11 antibodies (Cell Signaling Technology, Beverly, Mass.) were used to immunoprecipitate the genomic fragments. Five sequence fragments spanning six variants of CArG motifs (vCArG) within the promoter and a CArG motif in the first intron of FT were amplified from the immunoprecipitated genomic DNA. PCR products were visualized after 35 cycles using DNA purified from chromatin immunoprecipitated with antibodies against HA or Myc. Nonselected input DNA and Myc antibody-selected DNA were employed as PCR templates for the positive and negative controls, respectively. Quantitation of the enrichment of CArG motifs by the SVP-HA and FLC-HA proteins were performed on phosphoimager plates (Fujifilm BAS 2500; Fuji, Tokyo, Japan).

### Luciferase Reporter Assay

To generate the FT::LUC construct, we amplified the 1.8-kb of the FT promoter fragment using JH3096 (5′-TGAACACTAACATGATTGAATGACA-3′) (SEQ ID NO: 5) and JH2865 (5′-GATCTTGAACAAACAGGTGGT-3′) (SEQ ID NO: 6) and fused this to luciferase. The luciferase reporter constructs harboring the mutated vCArG motifs within the FT promoter were used as reporters to examine the effects of the vCArG motifs on the specific binding of SVP to the FT promoter. Site-directed mutagenesis was utilized to generate the FT::LUC constructs harboring the mutated vCArG motifs, using the QuickChange II XL Site-Directed Mutagenesis Kit (Stratagene, La Jolla, Calif.), in accordance with the manufacturer's instructions. Mutations introduced into the vCArG motifs in these constructs were verified via sequencing. SVP—with or without its MADS domain (35S::SVP-HA and 35S::SVP ΔM-HA, respectively)—was employed as an effector. A reporter and an effector were co-transfected into the protoplasts. The 35S::GUS construct was used as an internal control. Luciferase activities were normalized by GUS activities.

### Results

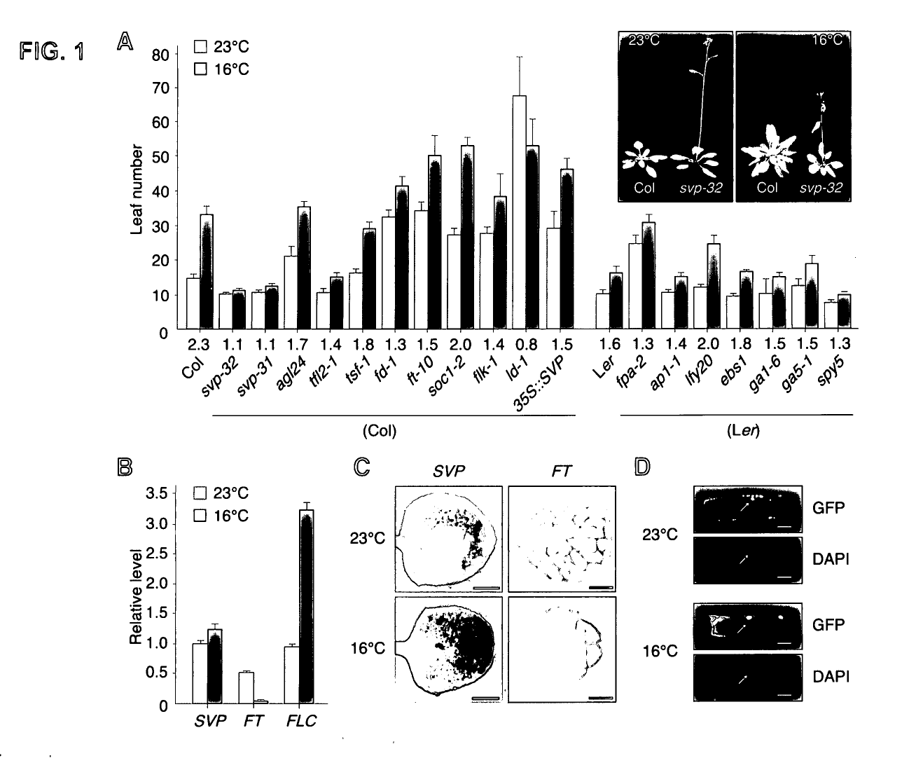

As a first step to determining the mechanism underlying the perception and transduction of ambient temperature signaling in plants, we assessed mutants in known flowering time genes for their insensitivity to changes in ambient growth temperature. Of the flowering time mutants tested, one with a lesion in svp was indeed insensitive to such changes. The flowering of the majority of these flowering time mutants was noticeably delayed at 16° C., with flowering time ratios (16° C./23° C.) ranging from 1.1 to 2.0 (FIG. 1A), the exception being Id-1. However, svp-31 and svp-32 mutants, the T-DNA alleles of SVP, manifested almost identical flowering times at 23° C. and 16° C. (FIG. 1A). svp mutants were early flowering, especially at 16° C., suggesting that a reduction in SVP activity significantly decreased plant response to lower temperature and that the loss of SVP activity would result in the loss of the effects of low temperature. In contrast, SVP overexpressor plants were late flowering, especially at 23° C., suggesting that overexpression of SVP can mimic the effect of low temperature.

Reduced FT expression was likely responsible for this late flowering phenotype of 35S::SVP plants. The weak temperature response seen in 35S::SVP plants can be explained by the differential expression of FT, such that FT expression at 23° C. was higher than that at 16° C. in 35S::SVP plants. SVP probably performs a non-redundant role in ambient temperature sensing, as loss of the function of AGL24, the closest homolog of SVP, did not induce temperature insensitivity (Yu, H. et al., 2002. Proc. Natl. Acad Sci. 99: 16336-16341).

Characterization of the pattern of SVP expression at different temperatures in wild-type plants by real-time PCR analyses revealed that SVP expression slightly increased in the leaf at 16° C. (FIG. 1B). In contrast, FT expression was strongly repressed in the leaf at 16° C. Histochemical β-glucuronidase (GUS) analysis detected both SVP and FT expression throughout the expanded leaves, but SVP expression was upregulated and FT expression was downregulated (FIG. 1C). Since this upregulation of SVP may not be significant in itself in explaining this dramatic downregulation of FT expression, it is possible that the post-transcriptional regulation of SVP or altered protein-protein interaction of SVP at the lower temperature may also be responsible for the reduction in FT transcription. Considering that SVP acts as a floral repressor, these data suggested that additional flower-inhibitory factors exist in the leaf at lower temperature. Subcellular localization analysis showed that the SVP-green fluorescence protein (GFP) fusion protein localized in the nucleus at both 16° C. and 23° C. (FIG. 1D). Taken together, these results indicate that SVP expression is weakly temperature-dependent, similar to the thermosensory genes of other species.

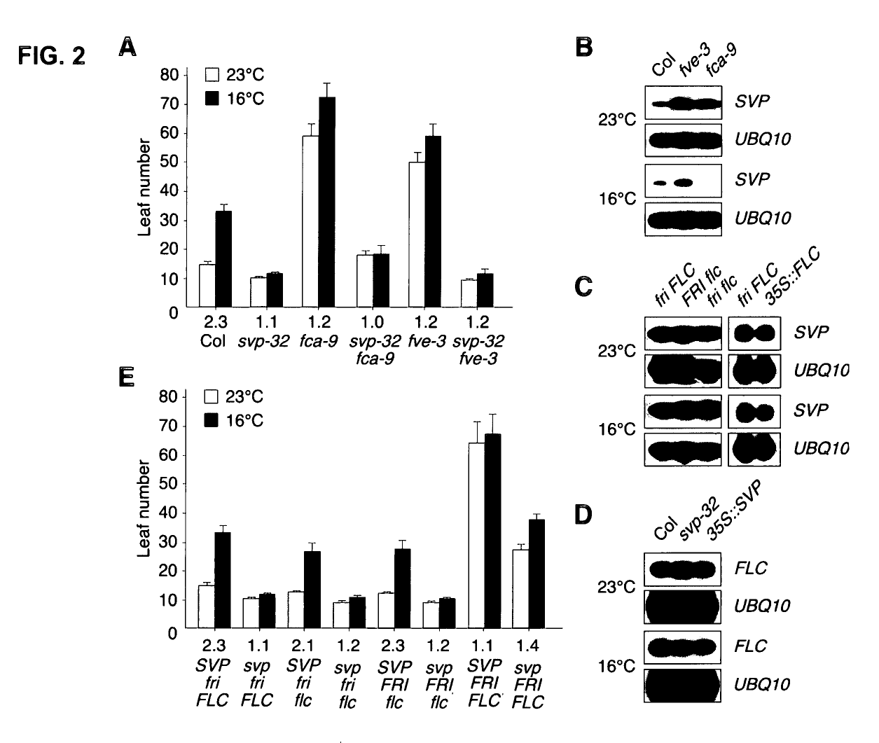

Ambient temperature is perceived via a genetic pathway (thermosensory pathway) that requires both FCA and FVE in Arabidopsis. An analysis of the genetic interaction of svp mutants with fca and fve mutants was conducted to ascertain whether or not SVP operates within the same genetic pathway as FCA and FVE. The late flowering phenotypes observed in the fca-9 and fve-3 mutants under long-day conditions were largely masked by the loss of SVP function (FIG. 2A), demonstrating that svp is epistatic to the fca and fve mutants.

In addition, the temperature insensitivity induced by the fca and fve mutations persisted even in the absence of SVP function, which suggests that SVP functions downstream of FCA and FVE within the thermosensory pathway and that SVP mediates temperature signaling. Consistent with this view, SVP expression was elevated in the fca and fve mutants (FIG. 2B) but not in other autonomous pathway mutants, and the flk and fpa mutants were temperature-sensitive (FIG. 1A). SVP expression, however, was regulated neither by vernalization nor by CONSTANS (CO), a central regulator of the long-day pathway. The observation that both the svp-32 mutants and wild type plants responded similarly to gibberellin (GA) treatment or to differing light conditions supports the premise that SVP functions primarily within the thermosensory pathway.

The genetic interaction of svp mutants with flc mutants was assessed in an attempt to determine whether or not SVP interacts with FLC, since FLC is an important regulator that mediates vernalization effects in the autonomous pathway, and both FLC and SVP function downstream of FCA and FVE (FIG. 2A,B). The results indicate that SVP acts independently of FLC at the transcriptional level within the thermosensory pathway: SVP expression was unchanged in the presence of functional alleles of FRI or FLC (FIG. 2C) and FLC expression remained unaffected by increases or reductions in SVP activity (FIG. 2D). Based on these results, we propose that SVP is very likely a thus-far unidentified repressor that mediates the temperature-dependent role of FCA and FVE. SVP appears to function, at least in part, downstream of FLC by modulating flowering time in response to ambient temperatures.

The flowering of fri flc, FRI flc, and FRI FLC mutants was accelerated by the svp-32 mutation (FIG. 2E). Conversely, the temperature responsiveness exhibited by the fri flc and FRI flc mutants disappeared in the absence of SVP function, thereby suggesting that SVP exerts its effects principally within the thermosensory pathway. Interestingly, at flowering, FRI FLC plants had a similar number of leaves at 23° C. and 16° C. (64 vs. 67 leaves), which was also found in fca and five mutants in which FLC levels were elevated even in the absence of functional FRI. A possible explanation of this flowering time phenotype of FRI FLC plants is that the floral repressive activity of FLC may be highly elevated in FRI FLC mutants and, consequently, further floral repression at 16° C. may be masked. Consistent with this premise, temperature responses were restored in fve flc and fca flc double mutants to a level similar to that shown by flc single mutants. Of particular interest is that the severe late flowering of FRI FLC plants was largely suppressed by the svp-32 mutation, suggesting that FLC requires SVP to inhibit flowering. Considering that the MADS box proteins are known to interact physically in a protein complex, a possible scenario to explain this suppression by the svp-32 mutation is that SVP and FLC proteins may interact in a complex during temperature signaling. This proposal is supported by recent findings that FLC is a component of a multimeric protein complex in vivo and that SVP interacts with several MADS box proteins.

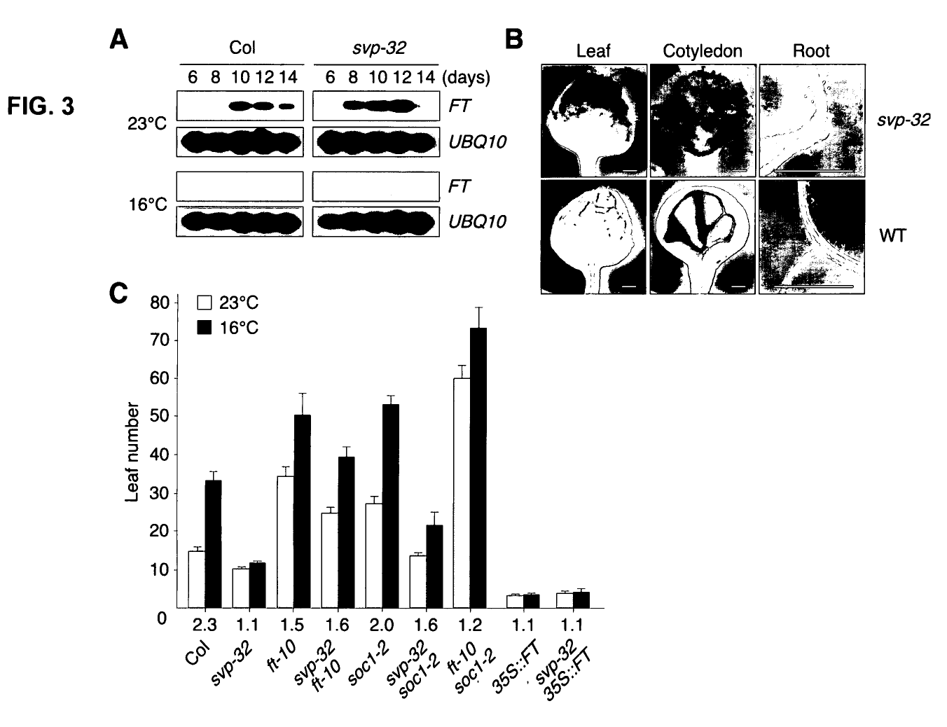

The conclusion that SVP functions as a floral repressor raises an important question: on which flowering time gene does SVP exert its negative effects in the transduction of ambient temperature signaling? An analysis of the expression levels of known flowering time genes in the svp mutants revealed that the expression levels of FT, a floral integrator, were substantially elevated in the svp-32 mutants at both 23° C. and 16° C. (FIG. 3A). A similar upregulation of FT in the svp-32 mutants was observed at a series of defined growth stages. These observations indicated that the thermosensory signaling pathway functions, at least in part, via FT.

A reporter assay, carried out to confirm the negative regulation of FT expression effected by SVP, revealed profound ectopic pFT::GUS expression in both the leaves and vascular root tissues of the svp-32 mutants (FIG. 3B). This suggests that SVP is required for the stable repression of FT in the ground tissues of the leaves of wild-type plants. Considering that FT is the major output of CO (Schmid, M. et al., 2003. Development 130: 6001-6012) and that FT mRNA is an important component of the long-distance signaling mechanism that triggers flowering, the early flowering phenotypes observed in the svp-32 mutants can be explained as follows: the absence of SVP activity induces the accumulation of FT mRNA in the leaf transportable to the shoot apex, thereby triggering floral development. Consistent with a role of FT downstream of SVP, the loss of FT function partially suppressed the early flowering of the svp-32 mutants, the constitutive expression of FT masked the phenotype in the svp-32 mutants (FIG. 3C), and FT expression was significantly reduced in 35S::SVP plants. Importantly, svp-32 ft-10 double mutants showed a weak temperature response, as did ft-10 mutants (flowering time ratio=1.6 vs. 1.5, respectively), although svp-32 single mutants showed temperature insensitivity. Similar phenotypic masking by temperature-sensitive mutants has been observed in fca flc and fve flc mutants. One possible scenario explaining why svp-32 ft-10 mutants were more responsive than svp-32 single mutants is that svp-32 mutants display a temperature-insensitive phenotype as the result of increased FT activity, the floral promoting effects of which are more profound at 16° C. When FT function is absent in the double mutants, the floral promoting effect by FT at 16° C. is not present and, therefore, temperature sensitivity may be restored to a level similar to that found in ft-10 single mutants. The observation that svp-32 ft-10 mutants were—albeit weakly—temperature sensitive indicates that the ambient temperature signaling mechanism of SVP requires FT and an additional downstream target(s). One possible target candidate is SUPPRESSOR OF OVEREXPRESSION OF CONSTANS 1 (SOC1), since the soc1-2 mutation additively reduced the temperature sensitivity of ft-10 mutants (flowering time ratio of ft-10 soc1-2 double mutants=1.2) (FIG. 3C), although soc1-2 single mutants responded to temperature changes. Consistent with this redundant role of FT and SOC1, neither theft-10 nor soc1-2 single mutations completely suppressed the early flowering phenotypes of the svp-32 plants (FIG. 3C). Rather, the early flowering of the svp-32 mutants is masked, in large part, by theft soc1 double mutation.

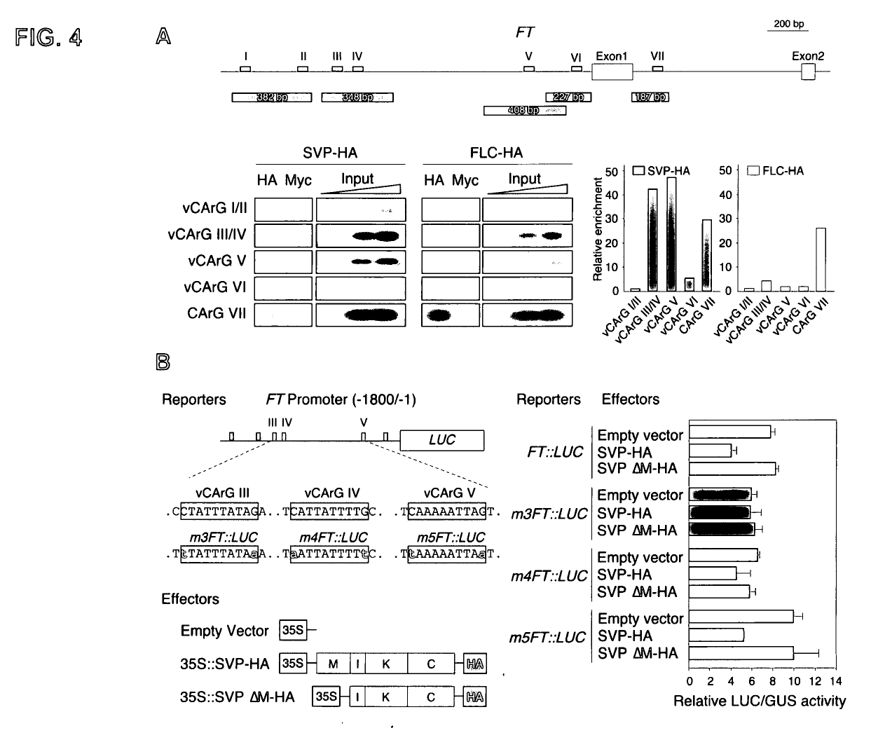

SVP is a member of the MADS box proteins, which function as transcriptional regulators via their DNA binding motifs. As such, it appears likely that the negative regulation of FT expression by the SVP protein can be achieved via direct binding to the FT sequence. This hypothesis was bolstered by the findings that the 1.8-kb promoter region of FT harbors six variants of CArG motifs (vCArG) (FIG. 4A), the consensus binding sequences of the MADS box proteins, and that the first intron of FT harbors a CArG motif to which FLC proteins directly bind. Chromatin immunoprecipitation (ChIP) assays using Arabidopsis protoplasts were carried out to evaluate this hypothesis. Using chromatin immunoprecipitated with HA antibodies, we detected amplified products from fragments harboring vCArG III/IV, vCArG V, and CArG VII (FIG. 4A), indicating that SVP and FLC proteins bind to these motifs in vivo.

The vCArG III/IV and vCArG V motifs were more efficiently precipitated by SVP-HA. The CArG VII motif, which is present in the first intron of FT, was strongly enriched by FLC-HA proteins, which is consistent with previous findings. This motif was also precipitated by SVP-HA proteins, but SVP's binding affinity appeared to be weaker than that of FLC. It therefore appears likely that SVP preferentially binds to the vCArG motifs of the FT promoter and that FLC preferentially binds to the CArG VII of the first intron of FT. As the vCArG III/IV and V motifs were observed to bind efficiently, we verified the direct binding of the SVP proteins to these motifs in vivo by conducting a transient expression assay in protoplasts transfected with SVP-HA proteins and FT-promoter-driven luciferase (LUC) reporters (FIG. 4B). An abundance of SVP protein (35S::SVP-HA) effected a reduction of FT::LUC activity. This reduction disappeared when SVP protein was used without its MADS domain (35S::SVP ΔM-HA), thereby indicating that the reduction in luciferase activity was induced by the binding of SVP to the FT promoter via the MADS domain. A subsequent assay aimed at assessing the ability of SVP-HA to repress the activity of an FT promoter harboring a mutation in the vCArG motif revealed that SVP-HA failed to reduce the expression of FT::LUC harboring mutations in vCArG III (m3FT::LUC). This result suggests that vCArG III is required for the SVP-mediated negative regulation of FT expression.

### EFFECT OF THE INVENTION

SVP gene isolated according to the present invention and SVP protein expressed from said gene can be useful for improving plant phenotypes that are related to flowering in plants and for searching a gene which is responsible for modulation of the flowering time in other plants. In addition, the present invention is advantageous in that, flowers and seeds can be produced in a short period of time by accelerating flowering time of plants, or vegetative growth can be continuously induced by delaying the flowering time of plants so that a productivity of useful plant parts such as leaves or stems can be improved.

What is claimed is:

1. SVP protein comprising amino acid sequences represented by SEQ ID NO: 2, which controls flowering time of plants originating from Arabidopsis.

2. SVP protein for controlling flowering time of plants according to claim 1, wherein the flowering time of plants is controlled in response to a change in ambient temperature.

3. A gene which encodes SVP protein according to claim 1.

4. The gene for controlling flowering time of plants according to claim 3, comprising nucleotide sequences represented by SEQ ID NO: 1.

5. The gene for controlling flowering time of plants according to claim 3, wherein the flowering time is controlled by the inhibition of the expression of FT (flowering locus T) gene, which accelerates the flowering, based on the binding of FT sequence to CArG motif.

6. A recombinant vector comprising the gene for controlling flowering time of plants according to claim 3.

7. A plant transformed with the recombinant vector according to claim 6.

8. A method of controlling flowering time of plants by using the gene according to claim 3.

9. The method according to claim 8, wherein the flowering time is delayed by overexpressing SVP gene in plants.

10. The method according to claim 8, wherein the flowering time is accelerated by inhibiting the expression of SVP gene in plants.

11. The method according to claim 8, wherein said plants are selected from a group consisting of food crops including rice, wheat, barley, corn, soybean, potato, red bean, oat and millet; vegetable crops including Arabidopsis thaliana, Chinese cabbage, radish, hot pepper, strawberry, tomato, watermelon, cucumber, cabbage, melon, pumpkin, scallion, onion and carrot; special crops including ginseng, tobacco, cotton, sesame, sugar cane, sugar beet, wild sesame, peanut and rapseed; fruits including apple, pear, date, peach, kiwi, grape, orange, persimmon, plum, apricot and banana; flowers including rose, gladiolus, gerbera, carnation, chrysanthemum, lily, and tulip; and feed crops including rye grass, red clover, orchard grass, alfalfa, tall fescue, and perennial rye grass.

12. A transformed plant of which flowering time is controlled by the method of claim 8.

13. A method for searching a protein or a gene for controlling flowering time of plants by carrying out an analysis using SVP protein of claim 1 or the gene encoding the same, wherein said analysis is selected from a group consisting of DNA chip method, protein chip method, polymerase chain reaction (PCR), Northern blot analysis, Southern blot analysis, enzyme-linked immunosorbent assay (ELISA) and 2D gel analysis.

14. A transformed plant of which flowering time is controlled by the method of claim 9.

15. A transformed plant of which flowering time is controlled by the method of claim 10.

16. A transformed plant of which flowering time is controlled by the method of claim 11.
